(19) United States
(12) Patent Application Publication
(10) Pub. No.: US 2024/0241140 A1
(43) Pub. Date: Jul. 18, 2024

(54) METHOD FOR EXAMINING PROGRESSION OF CHRONIC KIDNEY DISEASE IN SUBJECT, AND KIT

(71) Applicant: JICHI MEDICAL UNIVERSITY, Tokyo (JP)

(72) Inventor: Makoto KUROO, Shimotsuke-shi, Tochigi (JP)

(73) Assignee: JICHI MEDICAL UNIVERSITY, Tokyo (JP)

(21) Appl. No.: 18/557,131

(22) PCT Filed: Apr. 26, 2022
(86) PCT No.: PCT/JP2022/018869

§ 371 Date: Oct. 25, 2023

(30) Foreign Application Priority Data
Apr. 28, 2021 (JP) 2021-075828

(51) Int. Cl. G01N 33/70; G01N 33/68

(52) U.S. Cl. CPC G01N 33/70; G01N 33/6893; G01N 2800/347; G01N 2800/52

(57) ABSTRACT

The present invention includes a method for examining the progression of chronic kidney disease in a subject. The method includes measuring the creatinine concentration in the blood and urine of the subject and the phosphorus concentration in the urine. Next, an estimate for the phosphorous concentration in the primary urine on the basis of Formula (II):

$$ePTFp = \frac{Up}{Ucr} \times Scr \times 3.33 \qquad ( II )$$

is calculated, followed by comparing the estimate for the phosphorus concentration in the primary urine with the phosphorus concentration in the primary urine of a normal subject. Another aspect of the invention relates to a method for suppressing the progression of chronic kidney disease in a non-human mammal subject.

### TECHNICAL FIELD

One aspect of the present invention relates to a method for examining a progression of chronic kidney disease in a subject, and a kit. Another aspect of the present invention relates to a method for inhibiting a progression of chronic kidney disease in a non-human mammal subject.

### BACKGROUND ART

Chronic kidney disease, which is a type of kidney disorder, often occurs not only as a kidney disease, such as chronic glomerulonephritis, but also as a kidney complication of lifestyle-related diseases, such as diabetes and hypertension. The only way to inhibit a progression of chronic kidney disease is to identify a disease that causes kidney disorder in each patient and thoroughly treat the disease. As chronic kidney disease progresses to end-stage renal failure, renal replacement therapy (e.g., dialysis or kidney transplantation) becomes necessary. In such cases, the patient's activities of daily living (ADL) and/or prognosis deteriorate, even resulting in a heavy burden on the medical economy. Therefore, it is desired to develop a testing method for early detection of a progression of chronic kidney disease and a method for inhibiting a progression of chronic kidney disease.

A universally recognized pathology during a progression of chronic kidney disease is a decrease in the number of functional nephrons. A nephron is a functional unit of a kidney consisting of renal tubules and glomeruli. When the number of nephrons decreases, a compensatory mechanism works to increase an amount of substances excreted per nephron in a urine. The problem at this time is phosphorus. When the amount of phosphorus excreted per nephron increases, a phosphorus concentration in primary urine increases, and microparticles containing calcium phosphate crystals (hereinafter also referred to as “calciprotein particles”) are formed in a renal tubular lumen. Calciprotein particles are nanoparticles of a complex of calcium phosphate crystals and a serum protein, fetuin A. Calciprotein particles are known to have an activity of damaging renal tubular cells. Therefore, formation of calciprotein particles causes kidney disorder.

Calciprotein particles are also known to be present in blood. Therefore, formation of calciprotein particles also causes vascular diseases such as vascular endothelial dysfunction and vascular calcification, as well as non-infectious inflammation.

For example, Patent Literature 1 discloses a method for measuring calciprotein particles and assisting in testing for chronic kidney disease.

### CITATION LIST

### Patent Literature

Patent Literature 1: JP 6566697B

### SUMMARY OF INVENTION

### Technical Problem

As mentioned above, there is a need for examining methods for early detection of a progression of chronic kidney disease. However, clinical indicators for early detection of a progression of chronic kidney disease have not yet been identified.

Therefore, it is an object of the present invention to provide simple examining means for early detection of a progression of chronic kidney disease.

### Solution to Problem

The present inventor has conducted studies on various means for solving the above-described problem. The present inventor has found that an estimated primary urine phosphorus concentration, which is associated with formation of calciprotein particles that cause kidney disorder, can be calculated based on phosphorus and creatinine concentrations measured in conventional blood and urine tests. As a result, the inventor has found that a progression of chronic kidney disease can be easily examined by comparing the calculated value of the estimated primary urine phosphorus concentration in a target subject with a primary urine phosphorus concentration in a normal subject. Based on the obtained findings, the inventor has completed the present invention.

Specifically, the present invention encompasses the following aspects and embodiments.
(1) A method for examining a progression of chronic kidney disease in a subject, comprising the following steps:
   a measurement step of measuring a blood creatinine concentration, a urine creatinine concentration, and a urine phosphorus concentration in the subject;
   a calculation step of calculating an estimated primary urine phosphorus concentration (ePTFp) based on the following Formula (II):

$$ePTFp = \frac{Up}{Ucr} \times Scr \times 3.33 \qquad ( II )$$

where
   ePTFp represents the estimated primary urine phosphorus concentration,
   Up represents the urine phosphorus concentration,
   Ucr represents the urine creatinine concentration, and
   Scr represents the blood creatinine concentration; and
   a phosphorus concentration comparison step of comparing the estimated primary urine phosphorus concentration obtained in the calculation step with a primary urine phosphorus concentration in a normal subject.
(2) The method according to Embodiment (1), which further comprises measuring a concentration of a marker substance that is blood fibroblast growth factor-23 (FGF23) or urine L-type fatty acid binding protein (L-FABP) in the subject in the measurement step, and
   further comprises a marker substance concentration comparison step of comparing the blood FGF23 concentration or urine L-FABP concentration in the subject with a blood FGF23 concentration or urine L-FABP concentration in the normal subject.
(3) The method according to Embodiment (1) or (2), wherein the subject is a human.
(4) The method according to Embodiment (3), wherein the primary urine phosphorus concentration in the normal subject is 2.3 mg/dL.
(5) The method according to Embodiment (3) or (4), wherein the blood FGF23 concentration in the normal subject is 53 pg/mL.
(6) The method according to Embodiment (1) or (2), wherein the subject is a non-human mammal.
(7) The method according to Embodiment (6), wherein the non-human mammal is a feline.
(8) A method for inhibiting a progression of chronic kidney disease in a non-human mammal subject, comprising the following steps:
   a measurement step of measuring a blood creatinine concentration, a urine creatinine concentration, and a urine phosphorus concentration in the subject;
   a calculation step of calculating an estimated primary urine phosphorus concentration based on the following Formula (II):

$$ePTFp = \frac{Up}{Ucr} \times Scr \times 3.33 \qquad ( II )$$

where
   ePTFp represents the estimated primary urine phosphorus concentration,
   Up represents the urine phosphorus concentration,
   Ucr represents the urine creatinine concentration, and
   Scr represents the blood creatinine concentration;
   a phosphorus concentration comparison step of comparing the estimated primary urine phosphorus concentration obtained in the calculation step with a primary urine phosphorus concentration in a normal subject; and
   a treatment step of carrying out a treatment intervention against the progression of chronic kidney disease based on comparison results of the phosphorus concentration comparison step.
(9) The method according to Embodiment (8), which further comprises measuring a concentration of a marker substance that is blood fibroblast growth factor-23 (FGF23) or urine L-type fatty acid binding protein (L-FABP) in the subject in the measurement step,
   further comprises a marker substance concentration comparison step of comparing the blood FGF23 concentration or urine L-FABP concentration in the subject with a blood FGF23 concentration or urine L-FABP concentration in the normal subject, and
   further comprises carrying out a treatment intervention against the progression of chronic kidney disease based on comparison results of the marker substance concentration comparison step in the treatment step.
(10) The method according to Embodiment (8) or (9), wherein the treatment intervention is carried out by dietary therapy or drug therapy in the treatment step.
(11) The method according to any one of Embodiments (8) to (10), wherein the non-human mammal is a feline.
(12) A kit for examining a progression of chronic kidney disease in a subject, which comprises at least a measuring member for measuring a urine creatinine concentration and a urine phosphorus concentration in the subject, and a written instruction,
   wherein the measuring member is used for measuring the urine creatinine concentration and the urine phosphorus concentration in the subject, and
   wherein the written instruction describes procedures of a method for examining a progression of chronic kidney disease in a subject, the method comprising the following steps:
   a measurement step of measuring a blood creatinine concentration, a urine creatinine concentration, and a urine phosphorus concentration in the subject;
   a calculation step of calculating an estimated primary urine phosphorus concentration based on the following Formula (II):

$$ePTFp = \frac{Up}{Ucr} \times Scr \times 3.33 \qquad ( II )$$

where
   ePTFp represents the estimated primary urine phosphorus concentration,
   Up represents the urine phosphorus concentration,
   Ucr represents the urine creatinine concentration, and
   Scr represents the blood creatinine concentration; and
   a phosphorus concentration comparison step of comparing the estimated primary urine phosphorus concentration obtained in the calculation step with a primary urine phosphorus concentration in a normal subject.
(13) The kit according to Embodiment (12), wherein the method for examining a progression of chronic kidney disease
   further comprises measuring a concentration of a marker substance that is blood fibroblast growth factor-23 (FGF23) or urine L-type fatty acid binding protein (L-FABP) in the subject in the measurement step, and
   further comprises a marker substance concentration comparison step of comparing the blood FGF23 concentration or urine L-FABP concentration in the subject with a blood FGF23 concentration or urine L-FABP concentration in the normal subject.
(14) The kit according to Embodiment (12) or (13), wherein the subject is a human.
(15) The kit according to Embodiment (14), wherein the primary urine phosphorus concentration in the normal subject is 2.3 mg/dL.
(16) The kit according to Embodiment (14) or (15), wherein the blood FGF23 concentration in the normal subject is 53 pg/mL.
(17) The kit according to Embodiment (12) or (13), wherein the subject is a non-human mammal.
(18) The kit according to Embodiment (17), wherein the non-human mammal is a feline.
(19) A program for executing the method for examining a progression of chronic kidney disease in a subject according to any one of Embodiments (1) to (7).

### Advantageous Effects of Invention

According to the present invention, it becomes possible to provide simple examining means for early detection of a progression of chronic kidney disease.

The present description includes part or all of the contents as disclosed in the description and/or drawings of Japanese Patent Application 2021-075828, which is a priority document of the present application.

### BRIEF DESCRIPTION OF DRAWINGS

FIG. 1 shows an overview of phosphorus excretion in a renal nephron.

FIG. 2 shows results of a log-log plot analysis of a relationship between an estimated primary urine phosphorus concentration and a relative mRNA level of a marker substance gene or a concentration of a marker substance in serum in Study II. In FIG. 2, A represents a relationship between the estimated primary urine phosphorus concentration (horizontal axis, mg/dL) and a relative mRNA level of osteopontin, a renal tubular injury marker (vertical axis), and B represents a relationship between the estimated primary urine phosphorus concentration (horizontal axis, mg/dL) and a serum fibroblast growth factor-23 (FGF23) concentration (vertical axis, pg/mL).

FIG. 3 shows results of a log-log plot analysis of a relationship between an estimated primary urine phosphorus concentration and a concentration of a marker substance in urine or serum in Study III. In FIG. 3, A represents a relationship between the estimated primary urine phosphorus concentration (horizontal axis, mg/dL) and a urine L-type fatty acid binding protein (L-FABP) concentration (vertical axis, μg/gCre), and B represents a relationship between the estimated primary urine phosphorus concentration (horizontal axis, mg/dL) and a serum FGF23 concentration (horizontal axis, pg/mL).

FIG. 4 shows a transition of renal events occurring in patients in a low FGF23 group and patients in a high FGF23 group during a 5-year follow-up period in Study III. In FIG. 4, the horizontal axis is a follow-up period (days), and the vertical axis is a probability of no renal event.

FIG. 5 shows one embodiment of a method for examining a progression of chronic kidney disease in a subject according to one aspect of the present invention.

FIG. 6 shows a flowchart of an embodiment of a method for examining a progression of chronic kidney disease and a method for inhibiting a progression of chronic kidney disease in a subject according to one aspect of the present invention, in which the subject is a cat.

### DESCRIPTION OF EMBODIMENTS

### <1. Method for Examining Progression of Chronic Kidney Disease>

The present inventor has found that an estimated primary urine phosphorus concentration, which is associated with formation of calciprotein particles that cause kidney disorder, can be calculated based on phosphorus and creatinine concentrations measured in conventional blood and urine tests. As a result, the inventor has found that a progression of chronic kidney disease can be easily examined by comparing the calculated estimated primary urine phosphorus concentration in a target subject with a primary urine phosphorus concentration in a normal subject. Therefore, one aspect of the present invention relates to a method for examining a progression of chronic kidney disease in a subject (hereinafter also referred to as “examination method”).

In each aspect of the present invention, the “progression of chronic kidney disease” means an exacerbation of chronic kidney disease as a type of kidney disorder. In general, a case with findings of kidney disorder as determined by urine tests, blood tests, image diagnosis, or the like, and/or a glomerular filtration rate (GFR) below a specified value (usually 60 mL/min 1.73 m²) for more than several months is diagnosed as chronic kidney disease. It is also possible to judge a progression of chronic kidney disease on the basis of stage classification based on indicators such as creatinine concentration, symmetric dimethylarginine (SDMA) concentration, urine condition, and blood pressure (Japanese Society of Nephrology, ed., Clinical Practice Guideline for CKD 2012, Tokyo-Igakusha).

In the examination method in this aspect, the subject is preferably a human or non-human mammal (e.g., a warm-blooded animal such as a pig, dog, bovine, rat, mouse, guinea pig, rabbit, chicken, sheep, cat, monkey, baboon, or chimpanzee) subject or patient, more preferably a human patient or feline, still more preferably a human patient or cat. Cases of chronic kidney disease are increasing not only in humans but also in non-human mammals. It is known that felines, especially cats, have a higher incidence of chronic kidney disease than other mammals, such as humans and dogs, and there are many cases where chronic kidney disease progresses to chronic renal failure. One of the reasons for this is that felines, especially cats, have a smaller total number of nephrons in one kidney than other mammals (cats: 200,000 nephrons; dogs: 400,000 nephrons; humans: over 1 million nephrons). It has been reported that an incidence of chronic renal failure in all age groups was 0.9% in dogs and 1.6% in cats, and an incidence of chronic renal failure in a group aged 15 or older was 5.7% in dogs and 15.3% in cats (DiBartola, 1995). Therefore, by applying the examination method in this aspect to the subject, it is possible to find a progression of chronic kidney disease in an early stage by a convenient examination.

The examination method in this aspect comprises a measurement step, a calculation step, and a phosphorus concentration comparison step. In addition, the examination method in this aspect may optionally comprise a normal value determination step and a marker substance concentration comparison step. Each step will be described in detail below.

### [1-1. Measurement Step]

This step comprises measuring a blood creatinine concentration, a urine creatinine concentration, and a urine phosphorus concentration in the subject.

In this step, a blood creatinine concentration, a urine creatinine concentration, and a urine phosphorus concentration can be measured by blood tests and urine tests commonly performed in the art. It is preferable to determine a blood (e.g., serum) creatinine concentration by an enzymatic method. It is preferable to determine a urine creatinine concentration by an enzymatic method or colorimetric method. It is preferable to determine a urine phosphorus concentration by an enzymatic method, a colorimetric method, a molybdate direct method.

This step may further comprise measuring a concentration of a marker substance that is blood fibroblast growth factor-23 (FGF23) or urine L-type fatty acid binding protein (L-FABP). In the case of this embodiment, preferably, a blood FGF23 concentration is quantitatively determined by ELISA. Preferably, a urine L-FABP concentration is quantitatively determined by ELISA.

### [1-2. Calculation Step]

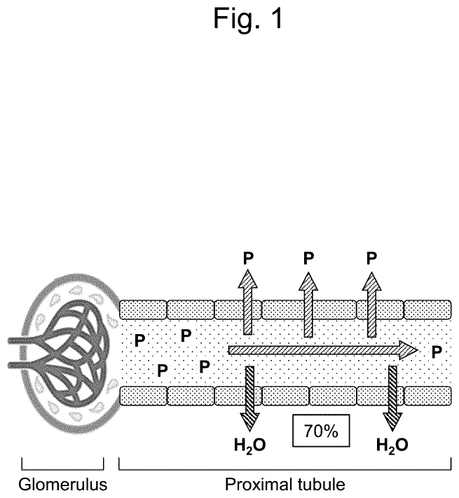

FIG. 1 shows an overview of phosphorus excretion in a renal nephron. As explained in Example (Study I) below, given that formation of calcium phosphate particles in renal tubular fluid is required for renal tubular injury, there should be a threshold for a primary urine phosphorus concentration (PTFp) in a proximal tubule above which calcium phosphate precipitation and tubular injury occur. However, it is technically difficult to collect primary urine from a living subject and directly measure a primary urine phosphorus concentration. The present inventor has found that an estimated primary urine phosphorus concentration can be calculated based on a blood creatinine concentration, a urine creatinine concentration, and a urine phosphorus concentration.

This step comprises calculating an estimated primary urine phosphorus concentration (ePTFp) based on the following Formula (II):

$$ePTFp = \frac{Up}{Ucr} \times Scr \times 3.33 . \qquad ( II )$$

In Formula (II),
ePTFp represents an estimated primary urine phosphorus concentration,
Up represents a urine phosphorus concentration,
Ucr represents a urine creatinine concentration, and
Scr represents a blood creatinine concentration.

As described above, a blood creatinine concentration, a urine creatinine concentration, and a urine phosphorus concentration in the subject can be measured by blood tests and urine tests commonly performed in the art. Therefore, the estimated primary urine phosphorus concentration, which can serve as an indicator of a progression of chronic kidney disease, can be calculated with a convenient examination by carrying out this step using the concentrations of substances obtained in the measurement step.

### [1-3. Normal Value Determination Step]

The examination method in this aspect may optionally comprise a normal value determination step of determining normal values of a primary urine phosphorus concentration and/or a blood or urine marker substance (e.g., blood FGF23 and urine L-FABP) concentration in a normal subject. In the examination method in this aspect, in a case in which normal values of the primary urine phosphorus concentration and/or the blood urine marker substance concentration in a normal subject have been determined, each step described below can be carried out using the normal values. However, in a case in which the normal values in a normal subject are not determined, the normal values in a normal subject can be obtained by carrying out this step.

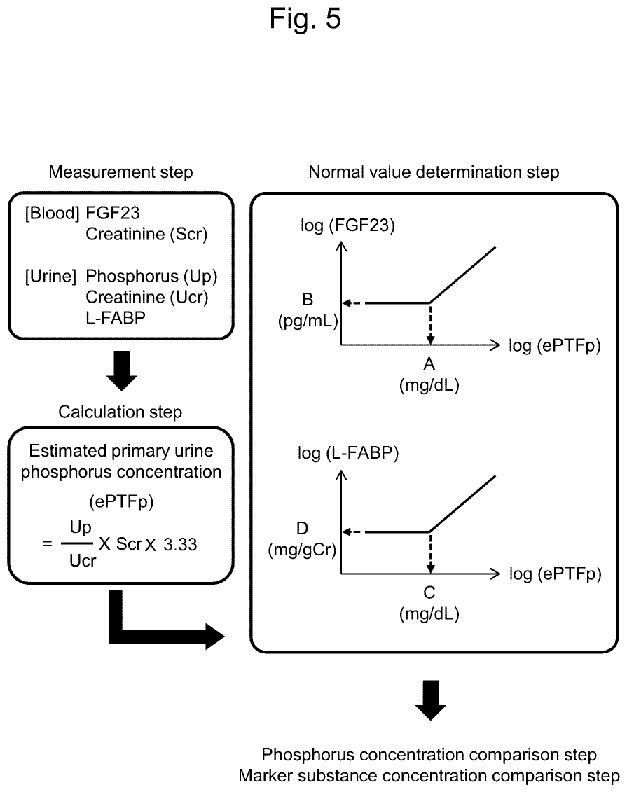

This step can be carried out by performing a log-log plot analysis of a relationship between an estimated primary urine phosphorus concentration (ePTFp) obtained from a plurality of subjects and concentrations of marker substances (e.g., blood FGF23 and urine L-FABP). In general, a relationship between an estimated primary urine phosphorus concentration and concentrations of marker substances is fitted to a biphasic linear regression. Concentrations of marker substances (e.g., blood FGF23 and urine L-FABP) are almost constant in a range where the estimated primary urine phosphorus concentration (ePTFp) is low; however, when the estimated primary urine phosphorus concentration (ePTFp) exceeds a specific threshold, they started to increase (FIG. 5). In this case, an almost constant values of marker substance concentration (B and D in FIG. 5) and specific thresholds of the estimated phosphorus concentration (ePTFp) (A and C in FIG. 5) can be determined to be normal values of the marker substance concentrations and the estimated phosphorus concentration in a normal subject.

The primary urine phosphorus concentration in a normal subject determined by the log-log plot analysis using different marker substances in this step is usually an identical value. For example, a primary urine phosphorus concentration corresponding to a blood FGF23 concentration in a normal subject determined by a log-log plot analysis of an estimated primary urine phosphorus concentration (ePTFp) and a blood FGF23 concentration is usually identical to a primary urine phosphorus concentration corresponding to a urine L-FABP concentration in a normal subject determined by a log-log plot analysis of the estimated primary urine phosphorus concentration (ePTFp) and a urine L-FABP concentration (A=C in FIG. 5).

Normal values of the primary urine phosphorus concentration and/or the concentrations of blood or urine marker substance (e.g., blood FGF23 and urine L-FABP) in a normal subject can be different depending on a type of subject. For instance, in a case in which the subject is a human, the primary urine phosphorus concentration in a normal subject is usually 5 mg/dL or less, for example, in a range of 0.1 to 3 mg/dL, especially 2.3 mg/dL. For instance, in a case in which the subject is a human, the blood (e.g., serum) FGF23 concentration in a normal subject is usually 100 pg/mL or less, for example, in a range of 10 to 80 pg/mL, especially 53 pg/mL. For instance, in a case in which the subject is a human, the urine L-FABP concentration in a normal subject is usually 10 μg/gCre or less, for example, in a range of 1 to 10 μg/gCre, especially 8.4 μg/gCre. For instance, in a case in which the subject is a feline, especially a cat, the primary urine phosphorus concentration in a normal subject is usually 700 mg/dL or less, for example, in a range of 50 to 700 mg/dL (Geddes, R. F. et al., The effect of feeding a renal diet on plasma fibroblast growth factor 23 concentrations in cats with stable azotemic chronic kidney disease. J Vet Intern Med, 27, 1354-1361, 2013).

### [1-4. Phosphorus Concentration Comparison Step]

This step comprises comparing the estimated primary urine phosphorus concentration obtained in the calculation step with a primary urine phosphorus concentration in a normal subject.

In this step, as the normal value of the primary urine phosphorus concentration in a normal subject, a preliminarily determined value may be used, or a value obtained by carrying out the normal value determination step described above may be used each time carrying out the examination method in this aspect.

In this step, the estimated primary urine phosphorus concentration obtained in the calculation step is compared with the primary urine phosphorus concentration in a normal subject. As a result of the comparison, in a case in which the estimated primary urine phosphorus concentration in a subject exceeds the primary urine phosphorus concentration in a normal subject, it can be judged that the subject is at a high risk of a progression of chronic kidney disease. Accordingly, by carrying out this step, a progression of chronic kidney disease can be detected at an early stage based on the estimated primary urine phosphorus concentration calculated based on the phosphorus and creatinine concentrations obtained by convenient tests.

### [1-5. Marker Substance Concentration Comparison Step]

The examination method in this aspect may further comprise a marker substance concentration comparison step of comparing a blood or urine marker substance concentration in a subject with a blood or urine marker substance concentration in a normal subject if desired.

In this step, for example, blood or urine marker substances are blood FGF23 and urine L-FABP.

In this step, as the normal value of the blood or urine marker substance concentration in a normal subject, a preliminarily determined value may be used, or a value obtained by carrying out the normal value determination step described above may be used each time carrying out the examination method in this aspect.

In this step, the blood or urine marker substance concentration obtained in the measurement step is compared with the blood or urine marker substance concentration in a normal subject. As a result of the comparison, in a case in which the blood or urine marker substance concentration in a subject exceeds the blood or urine marker substance concentration in a normal subject, it can be judged that the subject is at a high risk of a progression of chronic kidney disease. As described above, the normal value of the blood or urine marker substance concentration in a normal subject can be determined by performing a log-log plot analysis of a relationship between the estimated primary urine phosphorus concentration and the blood or urine marker substance concentration. Accordingly, by carrying out this step, a progression of chronic kidney disease can be detected at an early stage based on the estimated primary urine phosphorus concentration calculated based on the blood or urine marker substance concentration obtained by a convenient test and the phosphorus and creatinine concentrations obtained by convenient tests.

In the examination method in this aspect, each step may be performed only once or may be performed a plurality of times. In a case in which each step is carried out a plurality of times, it is preferable to judge a change in a risk of a progression of chronic kidney disease by carrying out the measurement step, the calculation step, and the phosphorus concentration comparison step (and optionally the marker substance concentration comparison step) again after carrying out the previous phosphorus concentration comparison step (and optionally the marker substance concentration comparison step). By performing each step a plurality of times in the examination method in this aspect, it is possible to detect a change in a risk of a progression of chronic kidney disease in an early stage by convenient means.

### <2. Program for Executing Method for Examining Progression of Chronic Kidney Disease>

Another aspect of the present invention relates to a program for executing a method for examining a progression of chronic kidney disease in a subject (hereinafter also referred to as “examination program”).

The examination program in this aspect can be used to execute the examination method in one aspect of the present invention on an analyzer (e.g., a computer, a smartphone, or a tablet terminal). The examination program in this aspect comprises a measurement step, a calculation step, and a phosphorus concentration comparison step. In addition, the examination program in this aspect may optionally comprise a normal value determination step and a marker substance concentration comparison step. The steps correspond respectively to the steps of the examination method in one aspect of the present invention described above.

The examination program in this aspect is usually used in a form stored in a storage medium such as a memory or a hard drive. Therefore, the examination program in this aspect may be provided in the form of a storage medium (e.g., a memory or a hard drive) that stores the examination program.

### <3. Kit for Examining Progression of Chronic Kidney Disease>

Another aspect of the present invention relates to a kit for examining a progression of chronic kidney disease in a subject (hereinafter also referred to as “examination kit”). The examination kit of this aspect comprises at least a measuring member for measuring a urine creatinine concentration and a urine phosphorus concentration in the subject and a written instruction.

In the examination kit of this aspect, the measuring member is used for measuring a urine creatinine concentration and a urine phosphorus concentration in the subject. It is preferable for the measuring member to quantitatively determine a urine creatinine concentration and a urine phosphorus concentration by the means described above. The measuring member is preferably a test strip for quantitatively determining a urine creatinine concentration and a urine phosphorus concentration using a colorimetric method. In the case of this embodiment, by comparing color of the colored test strip and colors of color samples, a urine creatinine concentration and a urine phosphorus concentration in the subject can be easily measured. For example, a urine creatinine concentration and a urine phosphorus concentration may be determined by visually comparing color of the colored test strip and colors of color samples. Alternatively, color of the colored test strip and colors of color samples are photographed by a camera, the color of the colored test strip is quantified from the obtained image data, and the quantified value is substituted into a calibration curve previously created by quantifying the colors of the color samples. Thus, the urine creatinine concentration and the urine phosphorus concentration may be calculated. Commercially available image analysis software may be used for quantifying color of the colored test strip. Alternatively, a program for executing image data capture, color quantification, and concentration calculation may be prepared.

In the examination kit of this aspect, the written instruction describes procedures of the examination method in one aspect of the present invention described above. The estimated primary urine phosphorus concentration, which can serve as an indicator of a progression of chronic kidney disease, can be calculated by measuring a urine creatinine concentration and a urine phosphorus concentration in the subject using the measuring member included in the examination kit of this aspect and executing the examination method in one aspect of the present invention using the obtained values based on the description by the written instruction.

It is known that in general the blood creatinine concentration (Scr) used in the calculation step does not significantly vary in a short period in the examination method in one aspect of the present invention described above, while the urine creatinine concentration (Ucr) and the urine phosphorus concentration (Up) vary depending on, for example, daily meal contents and/or drinking water intake. With the blood creatinine concentration (Scr) in the subject determined in advance, the estimated primary urine phosphorus concentration, which can serve as an indicator of a progression of chronic kidney disease, can be calculated to conveniently examine a progression of chronic kidney disease by regularly measuring the urine creatinine concentration (Ucr) and the urine phosphorus concentration (Up) in the subject using the measuring member included in the examination kit of this aspect and executing the examination method in one aspect of the present invention using the urine creatinine concentration (Ucr) and the urine phosphorus concentration (Up) obtained above and the blood creatinine concentration (Scr) determined in advance based on the description by the written instruction.

In one embodiment, it is preferable to use the examination kit in this aspect in combination with the examination program in one aspect of the present invention. It is more preferable to use the examination kit in combination with the examination program in one aspect of the present invention, and an analyzer (e.g., a computer, a smartphone, or a tablet terminal) with a storage medium (e.g., memory or hard drive) for storing the program for executing image data capture, color quantification, and concentration calculation for the measuring member (e.g., a test strip) described above, and a camera. In the case of this embodiment, for example, the subject can execute image data capture, color quantification, and concentration calculation for the colored measuring member (e.g., test strip) using the analyzer and further execute the examination program in one aspect of the present invention. By doing so, the subject can conveniently examine a progression of chronic kidney disease by oneself.

### <4. Method for Inhibiting Progression of Chronic Kidney Disease>

Another aspect of the present invention relates to a method for inhibiting a progression of chronic kidney disease in a subject (hereinafter also referred to as “inhibition method”).

In each aspect of the present invention, the expression “inhibiting a progression of chronic kidney disease” means delaying a progression of chronic kidney disease by treating symptoms of the chronic kidney disease.

In the inhibition method in this aspect, the subject is preferably a human or non-human mammal (e.g., a warm-blooded animal such as a pig, dog, bovine, rat, mouse, guinea pig, rabbit, chicken, sheep, cat, monkey, baboon, or chimpanzee) subject or patient, more preferably a human patient or feline, still more preferably a human patient or cat. In a specific embodiment, the subject is preferably a non-human mammal or feline exemplified above, more preferably a cat. By applying the inhibition method in this aspect to the subject, it is possible to find a progression of chronic kidney disease in an early stage by a convenient examination and inhibit the progression of chronic kidney disease.

The inhibition method in this aspect comprises a measurement step, a calculation step, a phosphorus concentration comparison step, and a treatment step. In addition, the inhibition method in this aspect may comprise a normal value determination step and a marker substance concentration comparison step. Each step will be described in detail below.

In the inhibition method in this aspect, the measurement step, the calculation step, the phosphorus concentration comparison step, the normal value determination step, and the marker substance concentration comparison step can be carried out in the same manner as each of the steps of the examination method in one aspect of the present invention described above.

### [4-1. Treatment Step]

This step comprises carrying out a treatment intervention against a progression of chronic kidney disease based on comparison results of the phosphorus concentration comparison step.

Preferably, the treatment intervention is carried out by dietary therapy or drug therapy in this treatment step. The dietary therapy can include, for example, restricting phosphorus intake based on a low-phosphorus diet. For example, when the subject is a human, the low-phosphorus diet is preferably a plant-based food or a food with low contents of food additives including phosphoric acid. For example, when the subject is a feline, especially a cat, the low-phosphorus diet is preferably a low-phosphorus pet food, a plant-based food, or a food with low contents of food additives including phosphoric acid. Examples of drug therapy include administering drugs such as a phosphorus adsorbent and a calciprotein (CPP) formation inhibitor. Preferably, the phosphorus adsorbent is calcium carbonate, a non-calcium containing phosphorus adsorbent, a Na⁺/H⁺ exchange transporter 3 (NHE3) inhibitor, or a sodium-dependent phosphate transporter 2b (Npt2b) inhibitor. Preferably, the CPP formation inhibitor is a bisphosphonate, magnesium, or zinc.

In the inhibition method in this aspect, each step may be performed only once or may be performed a plurality of times. In a case in which each step is performed a plurality of times, the same treatment intervention may be performed in a plurality of treatment steps, or different treatment interventions may be performed. Preferably, after the previous treatment steps are performed, the measurement step, the calculation step, and the phosphorus concentration comparison step (and optionally the marker substance concentration comparison step) are performed again to judge a change in a risk of a progression of chronic kidney disease; then based on the judgment, it is determined whether the same treatment intervention as the previous one is performed in the treatment steps of this time or a different treatment intervention is performed. By performing each step a plurality of times in the inhibition method in this aspect, it is possible to detect a change in a risk of a progression of chronic kidney disease in an early stage by convenient means and carry out treatment intervention in an early stage. Accordingly, a progression of chronic kidney disease, such as a transition to end-stage renal failure, can be inhibited.

### EXAMPLES

The present invention will be specifically described in the following examples. However, these examples are not intended to limit the scope of the present invention.

### <Study I: Calculation of Estimates of Primary Urine Phosphorus Concentration>

FIG. 1 shows an overview of phosphorus excretion in a renal nephron. Given that formation of calcium phosphate particles in renal tubular fluid is required for renal tubular injury, there should be a threshold for a primary urine phosphorus concentration (PTFp) in a proximal tubule above which calcium phosphate precipitation and tubular injury occur. It is technically difficult to collect primary urine from a cortico-medullary junction of a living mouse. Therefore, based on the following assumptions, it was decided to estimate PTFp from phosphorus and creatinine concentrations in blood and urine. Firstly, a phosphorus concentration in glomerular filtration is equivalent to a blood phosphorus concentration. Secondly, a proximal tubule reabsorbs 70% of filtered water under standard conditions (FIG. 1) (Baum M, and Quigley R. Proximal tubule water transport-lessons from aquaporin knockout mice. Am J Physiol Renal Physiol. 2005; 289(6): F1193-4). This causes a 3.33-fold increase in solute concentration. Thirdly, phosphorus reabsorption occurs exclusively in a proximal tubule (FIG. 1) (Blaine J, Chonchol M, and Levi M. Renal control of calcium, phosphate, and magnesium homeostasis. Clinical journal of the American Society of Nephrology: CJASN. 2015; 10(7): 1257-72). Finally, a phosphorus excretion fraction (FEp), defined as a ratio of phosphorus excretion to creatinine excretion, indicates a phosphorus fraction that is not reabsorbed in the proximal tubule. Therefore, it is estimated that a blood phosphorus concentration (Sp) and a value obtained by multiplying FEp by 3.33 reflect PTFp. The value is determined to be an estimated PTFp.

$$ePTFp = Sp \times FEp \times 3.33 = S p \times \frac{\frac{Up \times V}{Sp}}{\frac{Ucr \times V}{Scr}} \times 3.33 \qquad ( I )$$

In the formula,
ePTFp represents an estimated primary urine phosphorus concentration,
Sp represents a blood phosphorus concentration,
FEp represents a phosphorus excretion rate,
Up represents a urine phosphorus concentration,
Ucr represents a urine creatinine concentration,
Scr represents a blood creatinine concentration, and
V represents a 24-hour urine output.

As is apparent from Formula (I), since the blood phosphorus concentration Sp is canceled out in the formula, it does not affect the estimated primary urine phosphorus concentration. In other words, the estimated primary urine phosphorus concentration ePTFp is expressed by the following Formula (II).

$$ePTFp = \frac{Up}{Ucr} \times Scr \times 3.33 \qquad ( II )$$

To confirm the estimated primary urine phosphorus concentration expressed by Formula (II), it was compared with measured value of the primary urine phosphorus concentration. Table 1 lists measured values of a primary urine phosphorus concentration in proximal tubules collected from living SD rats by the micropuncture method and estimated primary urine phosphorus concentrations calculated based on Formula (II). The measured values of Sp, FEp, and primary urine phosphorus concentration are described in the literature (Bank, N., et al. Micropuncture study of renal phosphate transport in rats with chronic renal failure and secondary hyperparathyroidism. J Clin Invest 61, 884-894, 1978).

TABLE 1

| | | | Primary urine | |
|---|---|---|---|---|
| | | | phosphorus concentration | |
| | | | (mg/dL) | |
| | Sp | | Measured | Estimated |
| SD rat group | (mg/dL) | FEp | value | value |
| Control (normal diet) | 7.6 | 0.1 | 2.3 | 2.5 |
| Phosphorus load | 18.4 | 0.4 | 29.4 | 24.5 |
| (intravenous injection) | | | | |
| 5/6 nephrectomized | 7.3 | 0.6 | 11.7 | 14.6 |
| (normal diet) | | | | |

As shown in Table 1, the estimated primary urine phosphorus concentrations calculated based on Formula (II) were well consistent with the measured values of primary urine phosphorus concentration.

### <Study II: Relationship Between Phosphorus Concentration in Primary Urine and Renal Tubular Injury in Model Mouse>

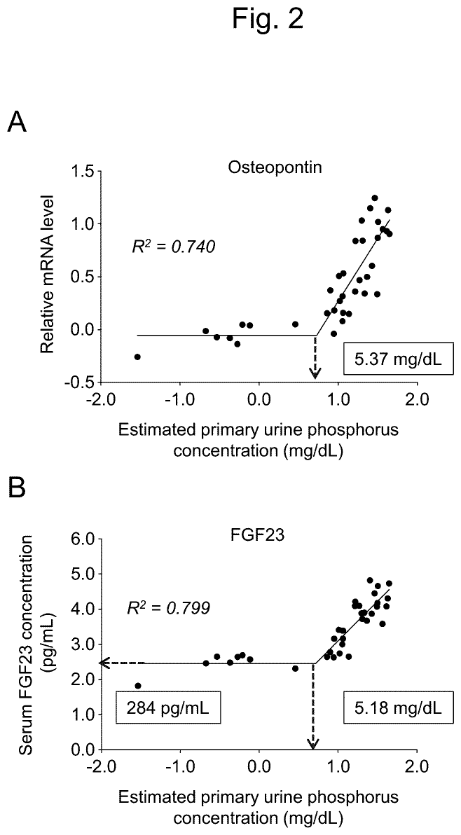

Normal mice and unilaterally nephrectomized mice were allowed to have a diet containing 0.35%, 1.0%, 1.5%, or 2.0% inorganic phosphate for 12 weeks. Relative mRNA levels of a given marker gene were determined by quantitative RT-PCR. The concentration of fibroblast growth factor-23 (FGF23) in serum was quantitatively determined by ELISA. The creatinine concentration in serum was quantitatively determined by an enzymatic method. The urine creatinine concentration was quantitatively determined by an enzymatic method. In addition, the urine phosphorus concentration was quantitatively determined by a molybdate direct method. The estimated primary urine phosphorus concentration was calculated based on Formula (II). FIG. 2 shows results of a log-log plot analysis of a relationship between the estimated primary urine phosphorus concentration and the relative mRNA level of a marker substance gene or the concentration of a marker substance in serum. In FIG. 2, A represents a relationship between the estimated primary urine phosphorus concentration (horizontal axis, mg/dL) and a relative mRNA level of osteopontin, a renal tubular injury marker (vertical axis), and B represents a relationship between the estimated primary urine phosphorus concentration (horizontal axis, mg/dL) and a serum FGF23 concentration (vertical axis, pg/mL).

As shown in FIG. 2, the relationship between the estimated primary urine phosphorus concentration and the expression level of the marker gene and the relationship between the estimated primary urine phosphorus concentration and the FGF23 concentration in serum were each fitted to a biphasic linear regression in which the slope of the first phase was zero. Specifically, the serum FGF23 concentration was almost constant in a range where the estimated primary urine phosphorus concentration (ePTFp) was low; however, when the estimated primary urine phosphorus concentration (ePTFp) exceeded 5.18 mg/dL, the serum FGF23 concentration started to increase (FIG. 2B). Consistent with the increase in serum FGF23 concentration, the expression level of osteopontin, a renal tubular injury marker, also began to increase (FIG. 2A). This was followed by increased expression of inflammatory markers such as monocyte chemoattractant protein-1 (MCP1) and transforming growth factor-β1 (TGFβ1) (data not shown). Increase in expression levels of fibrosis markers and decrease in renal function (decreased creatinine clearance) obviously occurred when the estimated primary urine phosphorus concentration reached approximately 10 mg/mL (data not shown).

The mechanism of renal tubular injury associated with increased FGF23 concentration is thought to be as follows. The increased phosphorus uptake and/or the decreased number of nephrons is accompanied by the increased phosphorus excretion per nephron to maintain phosphorus homeostasis. This requirement is consistent with the increased concentration of FGF23 that is a hormone that increases phosphorus excretion per nephron. However, the increased FGF23 concentration increases the primary urine phosphorus concentration, causing an increased risk for calcium phosphate particle formation in renal tubules. Calcium phosphate particles in renal tubules bind to Toll-like receptor 4 (TLR4) expressed on renal tubular cells and induce renal tubular injury. When nephrons are damaged by tubular injury, FGF23 increases further to compensate for the decrease in the number of nephrons unless the phosphorus uptake decreases, thereby triggering a cascade of deterioration leading to progressive nephron loss.

<Study III: Relationship Between Primary Urine Phosphorus Concentration and Renal Tubular Injury in Human Patients with Chronic Kidney Disease>

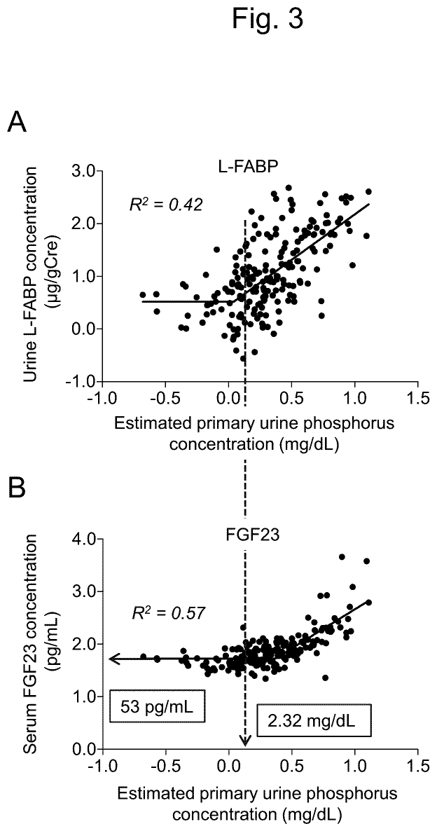

It was examined whether the relationship between the estimated primary urine phosphorus concentration and the FGF23 concentration in mice observed in Study II was also observed in humans. Non-dialyzed chronic kidney disease (CKD) patients (148 patients) at different stages were selected so as to measure the estimated primary urine phosphorus concentration and serum FGF23. L-type fatty acid binding protein (L-FABP) concentration in urine was quantitatively determined by ELISA. Serum FGF23 concentration was quantitatively determined by ELISA. FIG. 3 shows results of a log-log plot analysis of a relationship between the estimated primary urine phosphorus concentration and the urine L-FABP concentration and a relationship between the estimated primary urine phosphorus concentration and the serum FGF23 concentration. In FIG. 3, A represents a relationship between the estimated primary urine phosphorus concentration (horizontal axis, mg/dL) and the urine L-FABP concentration (vertical axis, μg/gCre), and B represents a relationship between the estimated primary urine phosphorus concentration (horizontal axis, mg/dL) and the serum FGF23 concentration (horizontal axis, pg/mL).

As shown in FIG. 3, in this cross-sectional study, the relationship between the estimated primary urine phosphorus concentration and the FGF23 concentration was fitted to a biphasic linear regression similar to Study II. The serum FGF23 concentration was almost constant in a range where the estimated primary urine phosphorus concentration (ePTFp) was low; however, when the estimated primary urine phosphorus concentration (ePTFp) exceeded 2.32 mg/dL, the serum FGF23 concentration started to increase (FIG. 3B). Similarly, the urine L-FABP concentration was almost constant in a range where the estimated primary urine phosphorus concentration (ePTFp) was low; however, when the estimated primary urine phosphorus concentration (ePTFp) exceeded 2.32 mg/dL, the urine L-FABP concentration started to increase (FIG. 3A). The results suggest that patients with estimated primary urine phosphorus concentrations exceeding 2.32 mg/dL and serum FGF23 concentrations exceeding 53 pg/mL have tubulointerstitial injury and progressive CKD (dotted and solid arrows in FIGS. 3A and B).

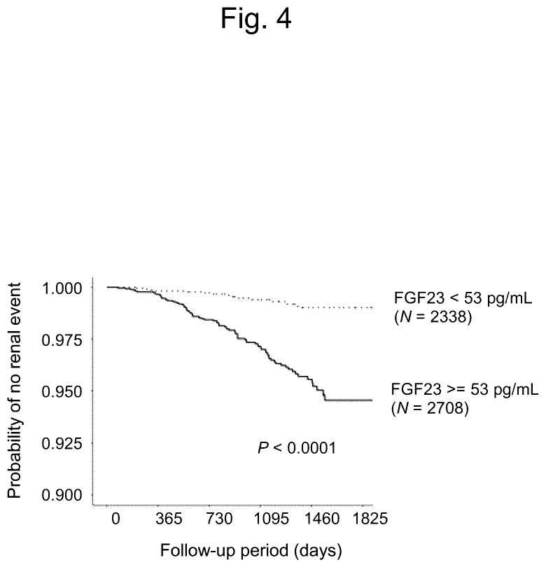

To test this possibility, a planned study was carried out to explore a relationship between serum FGF concentrations and incidental renal events using blood samples from stored specimens from the EMPATHY study at Jichi Medical University. The stored specimens were obtained from patients with hyperlipidemia and diabetic retinopathy but without progressive CKD (eGFR<30 mL/min/1.73 m²). The median FGF23 concentration was 54.7 pg/mL, with an interquartile range (IQR) of 44.0 pg/mL to 69.0 pg/mL. A total of 5039 patients were stratified into two groups, patients in the low FGF23 group and patients in the high FGF23 group, based on a baseline serum FGF concentration of 53 pg/mL. Renal events were defined by an introduction of renal replacement therapy or at least a 2-fold increase in serum creatinine concentration that occurred in 100 cases of patients during the 5-year follow-up period. Renal events accounted for 0.6% (14 out of 2336 cases of patients) in the low FGF23 group and 3.2% (86 out of 2703 cases of patients) in the high FGF23 group. FIG. 4 shows a transition of renal events occurring in patients in the low FGF23 group and patients in the high FGF23 group during the 5-year follow-up period. In FIG. 4, the horizontal axis is follow-up period (days), and the vertical axis is a probability of no renal event.

As shown in FIG. 4, the high FGF23 group was associated with increased renal events. Cox regression analysis showed that the high FGF23 group had a significantly higher risk for renal events compared to the low FGF23 group (hazard ratio (HR): 5.18; 95% confidence interval (CI): 2.94 to 9.11; P<0.001 by log-rank test). This relationship remained even after adjustment for age, sex, body mass index, and, importantly, serum creatinine (HR: 2.84; 95% CI: 1.57 to 5.13: P<0.001). The results of this study revealed that human patients with serum FGF23 concentrations exceeding 53 pg/mL are at high risk of the progression of chronic kidney disease.

### <Study IV: Examination and Treatment for Progression of Chronic Kidney Disease in Subjects>

FIG. 5 shows one embodiment of a method for examining a progression of chronic kidney disease in a subject according to one aspect of the present invention. As shown in FIG. 5, the blood creatinine concentration (Scr), urine creatinine concentration (Ucr), and urine phosphorus concentration (Up) in subjects are measured (measurement step). Marker substances, such as blood FGF23 and urine L-FABP concentrations, are optionally measured. An estimated primary urine phosphorus concentration (ePTFp) is calculated based on the following Formula (II) (calculation step):

$$ePTFp = \frac{Up}{Ucr} \times Scr \times 3.33 . \qquad ( II )$$

In a case in which a normal primary urine phosphorus concentration in a normal subject is not determined, the measurement and calculation steps are performed for a plurality of subjects, including a normal subject. The primary urine phosphorus concentration in a normal subject is determined by performing a log-log plot analysis of a relationship between the estimated primary urine phosphorus concentration (ePTFp) obtained from a plurality of subjects and concentrations of marker substances, such as blood FGF23 and urine L-FABP (normal value determination step). The primary urine phosphorus concentration in a normal subject determined by the log-log plot analysis using different marker substances in the normal value determination step is usually an identical value. For example, in FIG. 5, the primary urine phosphorus concentration A corresponding to the blood FGF23 concentration B in a normal subject determined by the log-log plot analysis of the estimated primary urine phosphorus concentration (ePTFp) and the blood FGF23 concentration is usually identical to the primary urine phosphorus concentration C corresponding to the urine L-FABP concentration D in a normal subject determined by the log-log plot analysis of the estimated primary urine phosphorus concentration (ePTFp) and the urine L-FABP concentration. Next, the estimated primary urine phosphorus concentration obtained in the calculation step is compared with a primary urine phosphorus concentration in a normal subject (phosphorus concentration comparison step). In addition, a marker substance concentration, such as a blood FGF23 concentration or urine L-FABP concentration, in a subject is compared with a marker substance concentration, such as a blood FGF23 concentration or urine L-FABP concentration, in a normal subject (marker substance concentration comparison step). For example, in a case in which the subject is a human, the primary urine phosphorus concentration is 2.3 mg/dl, and the blood FGF23 concentration is 53 pg/mL in a normal subject, as determined in the normal value determination step (Study III). Therefore, in a case in which the estimated primary urine phosphorus concentration in a human subject exceeds 2.3 mg/dL, which is the primary urine phosphorus concentration in a normal human subject in the phosphorus concentration comparison step, it can be judged that the subject is at a high risk of a progression of chronic kidney disease. Similarly, in a case in which the blood FGF23 concentration in a human subject exceeds 53 pg/mL, which is the blood FGF23 concentration in a normal human subject in the marker substance concentration comparison step, it can be judged that the subject is at a high risk of a progression of chronic kidney disease.

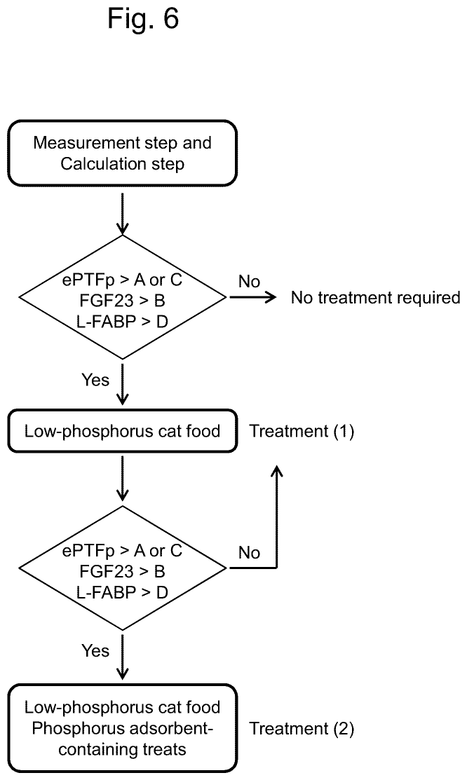

The method of the present invention can be applied to a variety of non-human mammals as well as humans. FIG. 6 shows a flowchart of an embodiment of the method for examining a progression of chronic kidney disease and the method for inhibiting a progression of chronic kidney disease in a subject according to one aspect of the present invention, in which the subject is a cat. As shown in FIG. 6, the measurement and calculation steps are performed on a feline subject. In a case in which a normal value A or C of the primary urine phosphorus concentration and the marker substance concentrations, such as blood FGF23 concentration B and urine L-FABP concentration D, are not determined for a normal feline subject, the normal value determination step may be performed. The estimated primary urine phosphorus concentration (ePTFp) obtained in the calculation step is compared with the primary urine phosphorus concentration A or C in a normal subject (phosphorus concentration comparison step). In addition, a marker substance concentration, such as a blood FGF23 concentration or urine L-FABP concentration, in a subject is compared with a marker substance concentration, such as a blood FGF23 concentration B or urine L-FABP concentration D, in a normal subject (marker substance concentration comparison step). In a case in which the estimated primary urine phosphorus concentration (ePTFp) in a feline subject exceeds the primary urine phosphorus concentration A or C (A and C values are generally identical) in a normal feline subject in the phosphorus concentration comparison step, it can be judged that the feline subject is at a high risk of a progression of chronic kidney disease. Similarly, in a case in which the blood FGF23 concentration or urine L-FABP concentration in a feline subject exceeds the blood FGF23 concentration B or urine L-FABP concentration D in a normal feline subject in the marker substance concentration comparison step, it can be judged that the feline subject is at a high risk of a progression of chronic kidney disease.

In a case in which it is judged that the feline subject is at a high risk of a progression of chronic kidney disease based on the comparison results of the phosphorus concentration comparison step or marker substance concentration comparison step, a treatment intervention is carried out against the progression of chronic kidney disease (treatment step). The treatment intervention may be carried out by, for example, limiting the phosphorus intake via feeding with a low-phosphorus cat food (treatment (1)). On the other hand, in a case in which the feline subject is at a low risk of a progression of chronic kidney disease, no special treatment is required.

After a particular period of time from the treatment intervention with treatment (1), the measurement step, the calculation step, the phosphorus concentration comparison step, and the marker substance concentration comparison step are performed. As a result, in a case in which the feline subject is still at a high risk of a progression of chronic kidney disease, another treatment intervention is carried out against the progression of chronic kidney disease (treatment step). Another treatment intervention may be carried out by, for example, administering a phosphorus adsorbent, in addition to limiting the phosphorus intake via feeding with a low-phosphorus cat food (treatment (2)). On the other hand, in a case in which it is judged that the risk of the progression of chronic kidney disease has decreased in the feline subject because of the treatment intervention with treatment (1), the treatment intervention with treatment (1) may be continued.

As described above, a progression of chronic kidney disease can be predicted by judging a risk of the progression of chronic kidney disease based on the method for examining a progression of chronic kidney disease in a subject according to the present invention. Therefore, by carrying out an early treatment intervention for a subject who is predicted to experience a progression of chronic kidney disease by the method of the present invention, the progression of chronic kidney disease, such as a transition to end-stage renal failure, can be inhibited.

### <Study V: Kit for Examining Progression of Chronic Kidney Disease in Subject>

Test strips for quantitatively determining urine creatinine and phosphorus concentrations using a colorimetric method were prepared. Color samples of test strips for the creatinine and phosphorus concentrations were photographed with a camera. Using image analysis software (Image J), colors of the color samples of the creatinine and phosphorus concentrations were digitized from the obtained image data so as to create a calibration curve for the creatinine and phosphorus concentrations. Urine from each of samples 1 to 3 was dropped on a test strip and photographed with a camera after 60 seconds. Using Image J, color of each test strip was quantitatively determined from the obtained image data. The urine creatinine and phosphorus concentrations were calculated by substituting the color values (signal intensities) of the test strips into the calibration curve. The results are shown in Tables 2 and 3.

TABLE 2

| | Specimen 1 | Specimen 2 |
|---|---|---|
| Color of phosphorus concentration | 42.60 | 55.69 |
| test strip (signal intensity, AU) | | |
| Phosphorus concentration (mg/dL) | 47.1 | 121.4 |

TABLE 3

| | Specimen | Specimen | Specimen |
|---|---|---|---|
| | 1 | 2 | 3 |
| Color of phosphorus concentration | 67.29 | 67.54 | 74.20 |
| test strip (signal intensity, AU) | | | |
| Phosphorus concentration (mg/dL) | 10.1 | 10.1 | 11.8 |

The present invention is not limited to Examples described above, but includes various modifications. For example, Examples described above are described in detail to explain the present invention in an easy-to-understand manner, and the present invention is not necessarily limited to having all the configurations described. Furthermore, it is possible to add, delete, and/or replace some of the configurations of each Example with other configurations.

All publications, patents, and patent applications cited in the present description are incorporated herein by reference in their entirety.

1. A method for examining a progression of chronic kidney disease in a subject, comprising the following steps:
a) measuring a blood creatinine concentration, a urine creatinine concentration, and a urine phosphorus concentration in the subject;
b) calculating an estimated primary urine phosphorus concentration (ePTFp) based on the following Formula (II):
$$ePTFp = \frac{Up}{Ucr} \times Scr \times 3.33 \qquad ( II )$$
wherein
ePTFp represents the estimated primary urine phosphorus concentration,
Up represents the urine phosphorus concentration,
Ucr represents the urine creatinine concentration, and
Scr represents the blood creatinine concentration; and
c) comparing the estimated primary urine phosphorus concentration obtained in the calculation step b) with a primary urine phosphorus concentration in a normal subject.

2. The method according to claim 1, which further comprises measuring a concentration of a marker substance that is blood fibroblast growth factor-23 (FGF23) or urine L-type fatty acid binding protein (L-FABP) in the subject in the measuring step a), and
further comprises a step of comparing the blood FGF23 concentration or urine L-FABP concentration in the subject with a blood FGF23 concentration or urine L-FABP concentration in the normal subject.

3. The method according to claim 1, wherein the subject is a human.

4. The method according to claim 3, wherein the primary urine phosphorus concentration in the normal subject is 2.3 mg/dL.

5. The method according to claim 3, wherein the blood FGF23 concentration in the normal subject is 53 pg/mL

6. The method according to claim 1, wherein the subject is a non-human mammal.

7. The method according to claim 6, wherein the non-human mammal is a feline.

8. A method for inhibiting a progression of chronic kidney disease in a non-human mammal subject, comprising the following steps:
a) measuring a blood creatinine concentration, a urine creatinine concentration, and a urine phosphorus concentration in the subject;
b) calculating an estimated primary urine phosphorus concentration based on the following Formula (II):
$$ePTFp = \frac{Up}{Ucr} \times Scr \times 3.33 \qquad ( II )$$
wherein
ePTFp represents the estimated primary urine phosphorus concentration,
Up represents the urine phosphorus concentration,
Ucr represents the urine creatinine concentration, and
Scr represents the blood creatinine concentration;
c) comparing the estimated primary urine phosphorus concentration obtained in the calculation step with a primary urine phosphorus concentration in a normal subject; and
d) carrying out a treatment intervention against the progression of chronic kidney disease based on comparison results of the phosphorus concentration comparison step.

9. The method according to claim 8, which further comprises measuring a concentration of a marker substance that is blood fibroblast growth factor-23 (FGF23) or urine L-type fatty acid binding protein (L-FABP) in the subject in the measuring step a),
further comprises a step of comparing the blood FGF23 concentration or urine L-FABP concentration in the subject with a blood FGF23 concentration or urine L-FABP concentration in the normal subject, and
further comprises carrying out a treatment intervention against the progression of chronic kidney disease based on the comparison results of the marker substance concentration comparison

10. The method according to claim 8, wherein the treatment intervention is carried out by dietary therapy or drug therapy.

11. The method according to claim 8, wherein the non-human mammal is a feline.

12. A kit for examining a progression of chronic kidney disease in a subject, which comprises a measuring member for measuring a urine creatinine concentration and a urine phosphorus concentration in the subject, and a written instruction, wherein the measuring member is used for measuring the urine creatinine concentration and the urine phosphorus concentration in the subject, and
wherein the written instruction describes procedures of a method for examining a progression of chronic kidney disease in a subject, the method comprising the following steps:
a measurement step of measuring a blood creatinine concentration, a urine creatinine concentration, and a urine phosphorus concentration in the subject;
a calculation step of calculating an estimated primary urine phosphorus concentration (ePTFp) based on the following Formula (II):
$$ePTFp = \frac{Up}{Ucr} \times Scr \times 3.33 \qquad ( II )$$
where
ePTFp represents the estimated primary urine phosphorus concentration,
Up represents the urine phosphorus concentration,
Ucr represents the urine creatinine concentration, and
Scr represents the blood creatinine concentration; and
a phosphorus concentration comparison step of comparing the estimated primary urine phosphorus concentration obtained in the calculation step with a primary urine phosphorus concentration in a normal subject.

13. The kit according to claim 12, wherein the method for examining a progression of chronic kidney disease
further comprises measuring a concentration of a marker substance that is blood fibroblast growth factor-23 (FGF23) or urine L-type fatty acid binding protein (L-FABP) in the subject in the measurement step, and
further comprises a marker substance concentration comparison step of comparing the blood FGF23 concentration or urine L-FABP concentration in the subject with a blood FGF23 concentration or urine L-FABP concentration in the normal subject.

14. The kit according to claim 12, wherein the subject is a human.

15. The kit according to claim 14, wherein the primary urine phosphorus concentration in the normal subject is 2.3 mg/dL.

16. The kit according to claim 14, wherein the blood FGF23 concentration in the normal subject is 53 pg/mL.

17. The kit according to claim 12, wherein the subject is a non-human mammal.

18. The kit according to claim 17, wherein the non-human mammal is a feline.

19. A program for executing the method for examining a progression of chronic kidney disease in a subject according to claim 1.
